(19) United States
(12) Patent Application Publication
(10) Pub. No.: US 2020/0327978 A1
(43) Pub. Date: Oct. 15, 2020

(54) METHODS, SYSTEMS, APPARATUSES AND DEVICES FOR FACILITATING DATA MANAGEMENT OF MEDICAL IMAGING DATA

(71) Applicant: George T. Fower, Costa Mesa, CA (US)

(72) Inventor: George T. Fower, Costa Mesa, CA (US)

(21) Appl. No.: 16/396,205

(22) Filed: Apr. 26, 2019

Related U.S. Application Data
(63) Continuation-in-part of application No. 16/381,698, filed on Apr. 11, 2019, now abandoned.
Application No. 16/381,698 is a continuation-in-part of application No. 16/380,575, filed on Apr. 10, 2019, now abandoned.

(51) Int. Cl. G16H 30/40; H04L 9/14

(52) U.S. Cl. CPC G16H 30/40; G06Q 2220/10; H04L 9/14

(57) ABSTRACT

A system of facilitating data management of medical imaging data is disclosed. Further, the system may include a communication device configured to receive the medical imaging data for a patient from a first party device on a server. Further, the communication device may be configured to receive a request from a second party device to retrieve the medical imaging data. Further, the communication device may be configured to securely transmit the medical imaging data to the second party device. Further, the system may include a storage device configured to store the medical imaging data on blockchain.

The current application claims a priority to a U.S. non-provisional application Ser. No. 16/380,575 filed on Apr. 10, 2019.

The current application also claims a priority to a U.S. non-provisional application Ser. No. 16/381,698 filed on Apr. 11, 2019.

### TECHNICAL FIELD

Generally, the present disclosure relates to the field of data processing. More specifically, the present disclosure relates to methods, systems, apparatuses and devices for facilitating data management of medical imaging data.

### BACKGROUND

In the US, all radiology studies are usually saved for 10 years. In 2006, there were over 400 million radiology studies performed in the US alone. This number has increased, perhaps 10 fold. In addition, government regulations require most studies be saved for 10 years, pediatric studies for 10-18 years, and mammograms forever. Conservatively, there is a need to store at least 10 billion studies in the US alone, and this number is growing annually. If each study is 200 MB (a low estimate), that is at least 2 petabytes of storage. Since studies are redundantly stored, the total amount of actual storage necessary is in the multiples of petabytes. Further, considering that the United States comprises only 12% of the worldwide imaging volume, the global storage requirement jumps 8-fold.

While this is a large number of radiologic images to store and audit, medical imaging is not only confined to diagnostic radiology; disciplines such as pathology, ophthalmology, dermatology, internal medicine, surgical sub-specialties, and dentistry are increasingly creating and storing digital images and videos.

At the same time that storage requirements increase, there is also a rising demand for rapid accessibility of medical imaging data. This is due to government edicts and societal demands for quick access to information. Transferring image data on printed or optical media has become insufficient for today's needs. Furthermore, the wide dissemination and accessibility of imaging data makes sense for an efficient medical ecosystem and sharing between healthcare organizations.

While storage and access to medical imaging data is a difficult enough technical problem, privacy regulations such as HIPAA have added additional complexity to any company developing medical imaging applications. And as data breaches at larger corporations such as Target, eBay, JP Morgan Chase, and even the Internal Revenue Service have eroded public trust in centralized storage of sensitive data, healthcare organizations are increasingly relying on their own hybrid cloud environments.

In summary, there is a need to store and disseminate large amounts of medical imaging data but bureaucratic and logistical obstacles, as well as lack of incentives, prevent an organization or organizations from doing so.

Therefore, there is a need for improved methods, systems, apparatuses and devices for facilitating data management of medical imaging data that may overcome one or more of the above-mentioned problems and/or limitations.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a method of facilitating data management of medical imaging data is disclosed. Further, the method may include receiving, using a communication device, the medical imaging data for a patient from a first party device on a server. Further, the method may include storing, using a storage device, the medical imaging data on blockchain. Further, the method may include receiving, using the communication device, a request from a second party device to retrieve the medical imaging data. Further, the method may include securely transmitting, using the communication device, the medical imaging data to the second party device.

According to some embodiments, a system of facilitating data management of medical imaging data is disclosed. The system may include a communication device configured to: receive the medical imaging data for a patient from a first party device on a server. Further, the system may include receive a request from a second party device to retrieve the medical imaging data. Further, the system may include securely transmit the medical imaging data to the second party device. Further, the system may include a storage device configured to store the medical imaging data on blockchain.

Further disclosed herein is a system of facilitating data management of medical imaging data. Further, the system may include a communication device configured to receive the medical imaging data for a patient from a first party device on a server.

Further, the communication device may be configured to receive a request from a second party device to retrieve the medical imaging data. Further, the communication device may be configured to securely transmit the medical imaging data to the second party device. Further, the system may include a storage device configured to store the medical imaging data on blockchain.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram representation of a system of facilitating data management of medical imaging data, in accordance with some embodiments.

FIG. 3 is a block diagram representation of the system that may further include a processing device configured to generate a symmetric key, in accordance with further embodiments.

FIG. 4 is a flowchart of a method of facilitating data management of medical imaging data, in accordance with some embodiments.

FIG. 5 is a flowchart of a method to facilitate providing an encrypted symmetric key, in accordance with further embodiments.

FIG. 6 is a flowchart of a method to facilitate functioning of RDGX blockchain, in accordance with some embodiments.

FIG. 7 is an exemplary representation of a system facilitating functioning of RDGX blockchain in accordance with some embodiments.

FIG. 8 is an exemplary representation of an RDGX blockchain, in accordance with some embodiments.

FIG. 9 is an exemplary representation of a smart contract code with a deployer public key and a smart contract public key, in accordance with some embodiments.

FIG. 10 is a block diagram of a computing device for implementing the methods disclosed herein, in accordance with some embodiments.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of data management of medical imaging data, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure includes systems and methods to facilitate data management for medical imaging data. Further, the present disclosure, in an instance, may include a project (may be referred to as Radiologex) which may feature following technological aspects and features, allowing massive improvements for current Radiological Images transfer, storage, and/or content input and reconstruction.

In some embodiments, the system may be implemented in 4 phases. Accordingly, the first phase, in an instance, may include safe and ultra-secure storage and archiving of medical radiology images, with unlimited storage (ZERO data caps on storage)—backed by a robust, Masternode operating, proof-of-stake, distributed ledger. Allowing an ultra-low cost, comprehensive solution to costly image transfer and storage.

Further, the second phase, in an instance, may include a Radiologex DEX (Decentralized Exchange), a platform utilizing an RDGX token in a direct P2P market exchange with BTC (bitcoin) pairing, featuring an Image Reading and Input content-based Marketplace, where Radiologists from worldwide may partake in content delivery. Further, both image producers (Imaging centers/Hospitals) and/or Accredited Radiologists, across the world, may near-instantly sell and/or purchase images to read and content to be immediately tended to whether in individual image files or bundle orders, drastically improving efficiency and turnaround time for high equality reports as well as providing a massive cost reduction across entire spectrum.

Further, the third phase, in an instance, may include deployment of an RDGX format. Further, the RDGX format, in an instance, may be an API based next generation Medical Imaging data format. Further, the RDGX format (and/or RDGX image file format), in an instance, may incorporate AI technology to allow for breakthroughs in Image Reconstruction.

Further, the fourth phase, in an instance, may incorporate four stages. Further, the first stage, in an instance, may include establishing a robust, ultra-secure, and/or powerfully fast CDN (content delivery network), the first of its kind in Medical Imaging, based upon the RDG protocol. Further, the second stage, in an instance, may include the CDN which may be configured to manifest a high-speed network of many GB's of connections per second, drastically improving image transfer, load, and Image acquisition times. Further stages associated with the fourth phase, in an instance, may include building up an RDGX protocol, and/or adding AI algorithms to allow instant on-demand Full Image Reconstruction capabilities including, 3D, 4D, Full Body advanced diffusion and perfusion-weighted, multi-contrast from single acquisition, ultra-noise reduction, advanced cardiac, and many other exciting new breakthroughs, which may not be currently possible on single station reconstruction. More excitingly, the RDGX protocol may be followed by an RDGX-VR that may be a full VR (Virtual Reality) ready image viewing and reconstruction platform.

Since it is possible to create an autonomous currency like Bitcoin, then it is also possible to use the same technology to create binding contracts, escrow transactions, third-party arbitration, or multiparty signatures that fulfill themselves instead of being enforced by a bank or governing body. Examples of smart contracts include a term life policy that automatically pays a death benefit on verification that a person dies or an auto loan that autonomously transfers a title to the owner when the principal is paid off.

In a properly designed smart contract, all property becomes smart property—it is encoded and bound to the blockchain in such a way that, using a unique identifier, it can be tracked, controlled, or exchanged.

Further, Radiologic organizations (such as but not limited to, hospital radiology departments, outpatient imaging centers etc.) may generate asymmetric encryption key pairs, store their private keys, and disseminate their public keys. As the radiologic organizations (RO) may create radiologic information (RI) including study images, scanned documents, reports etc., the RI may be encrypted using the RO's private key.

Each study is given a randomly assigned ID, and the ownership of the study (the RI) is encoded as a transaction in blockchain. This is analogous to mining a Bitcoin; the chain must be checked to see if the study ID exists on the chain before adding it. (The Bitcoin blockchain itself is not likely appropriate due to block size conflicts and the burdensome 10-minute block time. Ethereum or a new chain would be more ideal.)

While the clinical information in the RI is owned by the patient in a legal sense, patients will be encouraged to allow the ROs to be the custodians of their RI much the same way it is done currently. Of course, a patient could revoke ownership from the RO, removing it from the radiology blockchain. But, there is no incentive to do this; the patient would lose the benefits of portability and security.

Once encoded in the chain, the encrypted RI becomes smart property that is disseminated in a BitTorrent-like fashion to the entire network in the same way the block ledger is. ROs may choose to keep a copy of their RI on their servers—though this is not obligatory. Ideally, RO servers don't just hold their own studies; they store studies done all over the country—or the world. This is possible without needing to expand storage capacity because studies do not need to be backed up as they are extensively duplicated. Once RI has been disseminated the data is secure so long as the ROs private key is not compromised.

Patients and ROs can direct the flow of the RI by the creation and enforcement of smart contracts on the blockchain. For example, suppose a patient sees a new physician or is seen in an Emergency Room away from home. Assuming these new ROs (nRO) have created their own private/public key pairs, they submit a request to “lease” the patient's RI for a period of time.

The blockchain ledger specifies the owner of the RI, the originating RO (oRO). The nRO sends its public key to the oRO. The oRO responds by returning a randomly-generated and time stamped symmetric key encrypted using the nRO's public key. The oRO retrieves the imaging data from the disseminated store, decrypts it with their private key, re-encrypts it with the shared symmetric key, and digitally signs it before sending it to the nRO.

Once the nRO receives the cipher, it verifies the signature, proving authenticity of the data. Next, it decrypts the data using the shared symmetric key which it was able to decrypt using its own private key.

The transactions between the oRO, the nRO, and any intermediaries may be documented in the blockchain. This is crucial because there is a specified lease time whereby the time-stamped shared key will cease to function when the lease expires. If a nRO attempts to decrypt a cipher with an expired symmetric key, the oRO is notified as the transaction shows up on the blockchain ledger. At this point, the oRO can chose to renew the lease or report the nRO for inappropriately accessing RI it does not own.

Those entities responsible for storing the RI and the oRO for retrieving and re-encrypting the RI are paid a transaction fee—a stipulation outlined in the smart contract. This is done automatically with a cryptocurrency, and the transaction is also encoded in the blockchain.

In this scenario, ROs have an incentive to store and make available RI, and patients have their data widely available and secure. The system also saves money as the transaction costs would be more economical than overnight mail or repeating studies unnecessarily.

The system is not entirely “trustless” as the patient must still trust the RO, but it is an improvement on the system we have today. Only a compromise of the RO's private key would result in a third party being able to unlawfully access the information. Unfortunately, a leasing RO cannot be prevented from decrypting RI using an expired symmetric key; though, this would be documented in the public ledger, the blockchain, and repeat offenders could be caught easily.

Additionally, demographics can be removed from studies themselves, and patient information could be encoded in the blockchain instead. Thus, a compromise of RI would not necessarily be a breach of protected health information.

Patients may also elect to enter into smart contracts to have their anonymized RI entered into pools made available for research studies or teaching files, receiving compensation without risk of a confidentiality breach.

Further, in some embodiments, the system may be implemented with 3 phases. Accordingly, the first phase, in an instance, may include safe storage and archiving of medical radiology images, with unlimited storage (ZERO data caps on storage), which may be backed by a robust, Masternode ran, proof-of-stake, distributed ledger.

Further, the second stage, in an instance, may include deployment of RDG format that may be an API based, next generation Medical Imaging data format.

Further, the third Phase, in an instance, may incorporate 4 stages. Accordingly, the first stage, in an instance, may include establishing a robust, ultra-secure, and powerfully fast CDN (content delivery network), which may be first of its kind in Medical Imaging, based upon the RDG protocol.

Further, the second stage, in an instance, may include the CDN, which may be configured to manifest a high-speed network of many Gigabytes (GB s) of connections per second drastically improving image transfer, load, and Image acquisition times.

Further, the third stage, in an instance, may include building up the RDGX protocol, adding AI algorithms to allow instant on-demand Full Image Reconstruction capabilities including, 3D, 4D, Full Body advanced diffusion and perfusion-weighted, multi-contrast from single acquisition, ultra-noise reduction, advanced cardiac, and many other exciting new breakthroughs, which may not be currently possible on single station reconstruction. More excitingly, the RDGX protocol may be followed by an RDGX-VR that may be a full VR (Virtual Reality) ready image viewing and reconstruction platform.

Further, the fourth stage, in an instance, may include Radiologex DEX (Decentralized Exchange), which may be a platform utilizing a RDGX token in a direct P2P market exchange with BTC (bitcoin) pairing, featuring an Image Reading and Input content-based Marketplace, where Radiologists from worldwide may partake in content delivery.

Accordingly, the system and method may provide a next generation picture archiving and communication system (PACS) that may provide economical storage and/or convenient access to the medical imaging data (medical images) from multiple user devices. Further, the system and method, in an instance, may provide content delivery network (CDN) as core and/or along with p2p marketplace. Further, the system, in an instance, may be in collaboration with hospitals, imaging centers and/or third party applications for research, content and post-processing etc.

Radiologex:

Radiologex is a next-gen PACS platform powered by blockchain technology and the RDGX AI network. Further, the Radiologex allows radiological imagers to reach unprecedented speeds while affording unrivaled imaging clarity. Further, the Radiologex may be more intuitive, easier to operate, and is equipped with leading security features—all for less cost. (the Evolution of Teleradiology)

Radiologex is also a powerful content delivery platform that may be designed to not only replace current PACS system, but also current Radiology information system (RIS) and/or clinical information system (CIS), adding a unique and simplified marketplace that may deliver products, equipment, software, content, scheduling, bill settlements, research and collaboration and much more, in a quicker, more intuitive, user-friendly system, more advanced and provides more levels of security than any other Teleradiology platform ever-all at reduced cost.

As a full-service blockchain-based solution, Radiologex improves diagnosis quality across the spectrum. Further, the radiologex has been built from the ground up for today's medical professional, maximizing time, resources, and human capital. Further, the radiologex may be designed to act as a support mechanism for today's content provider and content interpreter.

Further, the Radiologex addresses existing shortcomings in the radiology field, including cost, speed, image quality, image interpretation, ease of use, collaboration, and security. Further, the radiologex is a finest tech integrating with teleradiology to offer a 21st-century diagnostic platform, and the first truly and entirely HIPPAA compliant network in the world with end-to-end encryption and full data custodianship.

Further, the radiologex, in an instance, may include features such as (but not limited to) User-Intuitive, Improved Workflow, Multi-Platform Support, WIN/MACOS/ANDROID/IOS Desktop and Mobile, Advanced Voice Dictation Capabilities, Multi-Language Support, State-of-the-Art Blockchain and AI-powered platform, Ultra-Secure Record Keeping and Limitless Storage, Image Reporting Marketplace/Equipment Marketplace, Content Marketplace-Software, apps, options, training, protocols, and more., Instant Payment Settlement, Advanced Post-Processing, RDGX AI-Powered VR Capabilities—with third party AI support, Complete RIS/CIS Suite In One Platform

Further, Radiologex makes navigation and diagnostics painless with a seamless platform. Accomplish more tasks in less time aided by powerful tools—advanced voice dictation and auto dictation capabilities with the ability to perform inversions, measurements, full reports, processing, transfers, and secure storage—without the clutter. Radiologex users may enjoy a highly responsive system with fluid workflow and user-friendly settings. WIN/MACOS/ANDROID/IOS compatible—sign up free and easily use PACS services in minutes.

Further, the radiologex, in an instance, may allow the user to view a crisp, coherent image and get the most of every scan with little to no compression or detail loss.

Further, the radiologex, in an instance, may allow the user to find enhanced DICOM and a dual-blockchain powered RDGX AI network, ushering in a new era of ultra-high quality MRI, CT, X-Ray, Ultrasound, Mammo, Nuclear Medicine, and other image modality data retrieval, tools, secure storage, reporting, and much more.

Further, the radiologex, in an instance, may allow features such as Advanced post-processing features and VR capabilities powered by RDGX AI*, reinforced by an advanced RIS/CIS suite, all in one platform.

Further, the radiologex, in an instance, may allow Low Overhead, Secure Payments. Further, in some embodiments, the radiologex, in an instance, may be an ultra-secure payment platform featuring instant payment settlement, even across borders, with exceptionally low fees—all through the RDGX token and blockchain payment system. Reduce accounting overhead and logjams while benefiting from overall savings of approximately 50% to 90% from currently available incumbent PACS and RIS solutions.

Further, advanced tokenization model allows the RDGX token to be used for all features and services, including payment settlements, and includes staking, rewards system and personal reputation scoring systems, all allowing significant cost offsets within the ecosystem.

Further, subscription packages and Pay-As-You-Go plans may be available and accept all major credit cards and ACH debits with fully-integrated USD/EURO/and BTC support. Further, the user may be provided choice in payment methods and currencies.

Further, in some embodiments, 5% of all net profit may be donated quarterly and distributed transparently to qualified, user-approved medical and health charities through the Radiologex Foundation using a real-time donation fund tracking tool.

Further, the radiologex, in an instance, may provide decentralized and/or Safe Storage. Radiologex may be powered by a proprietary third-generation, cutting edge, dual-blockchain system with end-user controlled privacy and blazing fast, ultra-secure, immutable data storage equipped with high-definition image retrieval, viewing, and reporting. Further, Radiologex may be fully HIPPA compliant with permanent safe storage across a decentralized platform—a giant leap forward from current cloud-based PACS solutions.

Further, in some embodiments, Radiologex may work under the protection of a double-encryption mechanism and may be free from costly and antiquated hardware and servers. Step into the high-powered ecosystem that instantly connects a user's entire radiology and medical world, delivering you content, tools, and services to help the user store, transfer, read, report, process, transact, communicate, and collaborate—all on a closed, permission-based enterprise level private blockchain.

Further, Radiologex makes the user to be the sole custodian of their patient's data with no fail points. Control and monitor personnel access with view tracking and enjoy instant data retrieval that acts as a bridge between medical entities and patients with powerful, full end-to-end encryption, and the most advanced security available cryptographically signed, the strongest true HIPAA compliance available.

Further, Radiologex, in an instance, may facilitate getting reports delivered faster. A Radiologex Direct Marketplace links qualified Content Producers with Content Interpreters (Radiologists), who have been verified with KYC procedures.

Further, in some embodiments, equipment marketplace with direct P2P support using an advanced proprietary RD SMART CONTRACTS™, perform and execute simple or complex transactions easily and seamlessly, track equipment titles instantly, conduct sales without costly intermediaries or brokers, purchase supplies and consumables using the network's purchasing power for vast cost savings, collaborate, research, seek jobs and attain talent on our job's marketplace, and access direct content including 3rd party postprocessing, software, apps, training, and AI support with the RDGX SDK™, complete service section delivers the user DIRECT service support to service providers and engineers, avoiding delays and costly “single choice' service options.

Further, the Radiologex, in an instance, may be used in English, Spanish, French, German, Mandarin, and Russian and more languages. Live worldwide tech support may always be available via chat, call, Skype, telegram, email, and SMS—every minute of the day, all year long.

Radiologex is conceived, designed, and built as an all-inclusive, powerful platform to realign the teleradiology industry by offering comprehensive solutions. With Radiologex, a user may communicate with millions of users across the globe, all on one network and one platform. Collaborate, train, learn, buy, trade, verify assets and titling (such as equipment), perform simplified med legal processes, buy products for less with negotiated user prices for supplies, transact business with evolved smart contracts in a trustless blockchain system, and access a P2P jobs and a service marketplace that is open around the clock. Save time and money in the largest, most innovative, and most secure radiology network in the world.

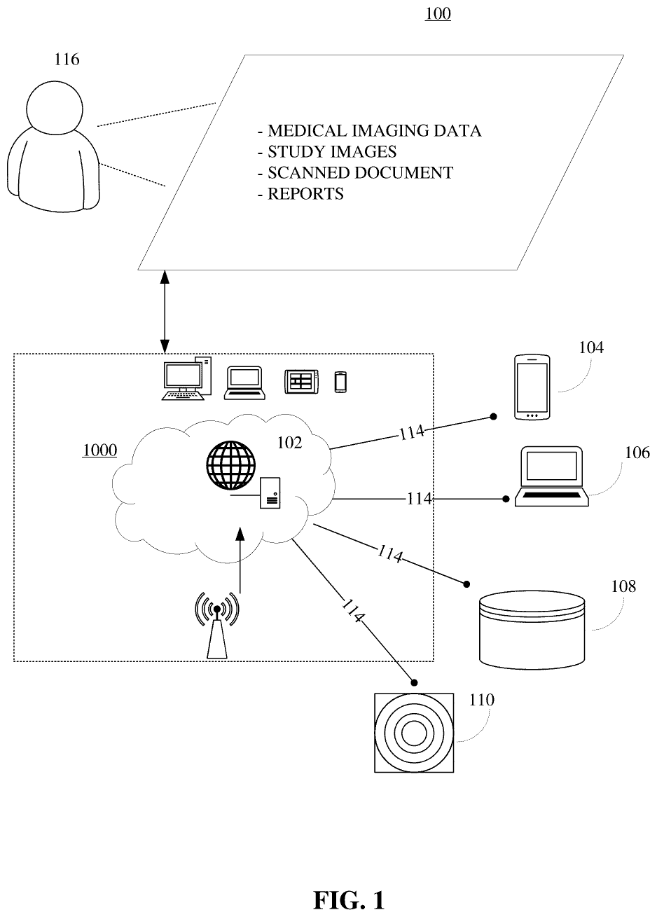

FIG. 1 is an illustration of an online platform 100 consistent with various embodiments of the present disclosure. By way of non-limiting example, the online platform 100 to facilitate data management of medical imaging data may be hosted on a centralized server 102, such as, for example, a cloud computing service. The centralized server 102 may communicate with other network entities, such as, for example, a mobile device 104 (such as a smartphone, a laptop, a tablet computer etc.), other electronic devices 106 (such as desktop computers, server computers etc.), databases 108, and sensors 110 over a communication network 114, such as, but not limited to, the Internet. Further, users of the online platform 100 may include relevant parties such as, but not limited to, end users, administrators, service providers, service consumers and so on. Accordingly, in some instances, electronic devices operated by the one or more relevant parties may be in communication with the platform.

A user 116, such as the one or more relevant parties, may access online platform 100 through a web based software application or browser. The web based software application may be embodied as, for example, but not be limited to, a website, a web application, a desktop application, and a mobile application compatible with a computing device 1000.

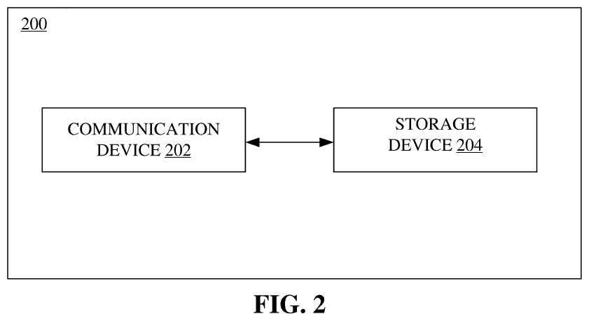

FIG. 2 is a block diagram representation of a system 200 of facilitating data management of medical imaging data, in accordance with some embodiments. Accordingly, the medical imaging data, in an instance, may be any medical data (such as study images, scanned documents, reports etc.) in a digital form, which may be used to diagnose and/or treat diseases within the user's body. For instance, the medical imaging data may include medical data (such as digital images and/or videos) related to disciplines such as, but not limited to, diagnostic radiology, pathology, ophthalmology, dermatology, internal medicine, surgical sub-specialties, and/or dentistry etc. Further, in some embodiments, the medical imaging data may include (but not limited to) radiologic studies, pediatric studies, mammograms etc. Further, the system 200 may include a communication device 202 configured to receive the medical imaging data for a patient from a first party device on a server. Further, the first party device, in an instance, may be any device and/or medical instrument that may be used for radiology in order to generate the medical imaging data for the patient. For instance, the first party device may include instruments such as, but not limited to, Ultrasonography machine, X-ray, Echocardiography machine, Computer axial tomography scan (CAT/CT scan), Magnetic resonance imaging (MRI), linear accelerator, functional magnetic resonance imaging (fMRI), Positron emission tomography(PET Scan), Interventional radiology, Brachytherapy apparatus etc. Further, in some embodiments, the first party device may be operated by an Originating Radiologic Organization (oRO). Further, the oRO, in an instance, may be any radiologic organization that may be configured to operate the first party device in order to generate the medical imaging data for the patient. For instance, the originating radiologic organization may include, but not limited to, hospital radiology departments, outpatient imaging centers etc. Further, in some embodiments, the medical imaging data received from the first party device may be encrypted using a first private key associated with the first party device. For instance, the medical imaging data may be encrypted by the first party device using the first private key before sending the medical imaging data to the server. Further, the first private key, in an instance, may only be known to the oRO that may be configured to operate the first party device.

Further, the communication device 202 may be configured to receive a request from a second party device to retrieve the medical imaging data. Further, the second party device, in an instance, may be an IoT based user device that may be configured to communicate with the server in order to submit the request to the oRO, for instance, to “lease” the medical imaging data for the patient for a period of time. In some embodiments, the second party device may be operated by one of a New Radiologic Organization (nRO), a physician, a researcher and a medical institution. Further, the nRO, in an instance, may be any radiologic organization that may wish to access the medical imaging data for the patient from the oRO. Further, in some embodiments, the request received from the second party device may include a second public key associated with the second party device. Further, the second public key, in an instance, may be a cryptographic key that may be used by the nRO to request the oRO to provide the medical imaging data associated with the patient.

Further, the communication device 202 may be configured to securely transmit the medical imaging data to the second party device.

Further, the system 200 may include a storage device 204 configured to store the medical imaging data on blockchain.

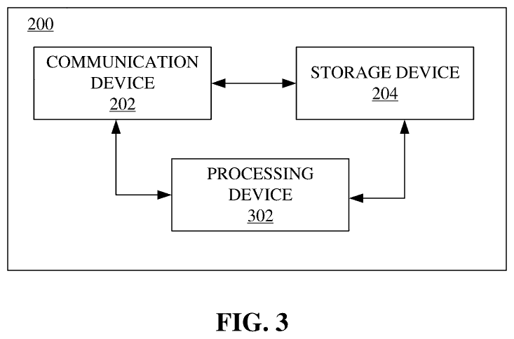

FIG. 3 is a block diagram representation of the system 200 that may further include a processing device 302 configured to generate a symmetric key, in accordance with further embodiments. Further, the symmetric key, in an instance, may be randomly generated and/or may be time-stamped. Further, the processing device 302 may be configured to encrypt the symmetric key using the second public key to obtain an encrypted symmetric key.

Further, the communication device 202 may be configured to send the encrypted symmetric key to one or more of the first party device and the second party device. Further, once the second party device may receive the encrypted symmetric key, the second party device may verify the signature, proving authenticity of the medical imaging data.

Further, in some embodiments, the communication device 202 may be configured to send the medical imaging data to the first party device. Further, the first party device may decrypt the medical imaging data with the first private key, may re-encrypt the medical imaging data with the symmetric key to obtain a re-encrypted imaging data, may digitally sign the re-encrypted imaging data and/or may send the re-encrypted imaging data to the second party device. Further, in some embodiments, the re-encrypted imaging data may be sent to the second party device via the server.

Further, in another embodiment, the server may decrypt the medical imaging data with the first private key, may re-encrypt the medical imaging data with the symmetric key to obtain a re-encrypted imaging data, may digitally sign the re-encrypted imaging data and/or may send the re-encrypted imaging data to the second party device.

Further, in some embodiments, the second party device may decrypt the encrypted symmetric key using a second private key associated with the second party device to obtain a decrypted symmetric key. Further, in some embodiments, the second party device may decrypt the medical imaging data using the decrypted symmetric key.

Further, in some embodiments, the processing device 302 may be configured to record one or more transactions in blockchain. Further, the one or more transactions may include receiving the request from the second party device, transmitting the medical imaging data to the second party device, sending the re-encrypted imaging data to the second party device and/or sending the encrypted symmetric key to one or more of the first party device and the second party device.

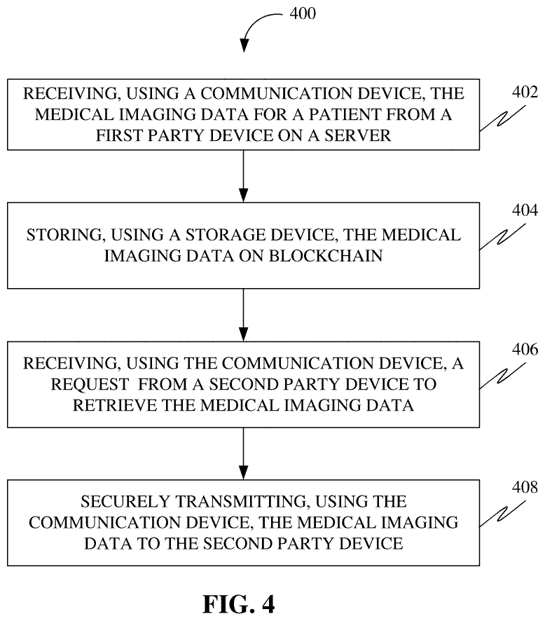

FIG. 4 is a flowchart of a method 400 of facilitating data management of medical imaging data, in accordance with some embodiments. Accordingly, at 402, the method 400 may include receiving, using a communication device (such as the communication device 202), the medical imaging data for a patient from a first party device on a server. Further, in some embodiments, the first party device may be operated by an Originating Radiologic Organization (oRO). Further, in some embodiments, the medical imaging data received from the first party device may be encrypted using a first private key associated with the first party device.

Further, at 404, the method 400 may include storing, using a storage device (such as the storage device 204), the medical imaging data on blockchain.

Further, at 406, the method 400 may include receiving, using the communication device, a request from a second party device to retrieve the medical imaging data. Further, in some embodiments, the second party device may be operated by one of a New Radiologic Organization (nRO), a physician, a researcher and a medical institution. In some embodiments, the request received from the second party device may include a second public key associated with the second party device.

Further, at 408, the method 400 may include securely transmitting, using the communication device, the medical imaging data to the second party device.

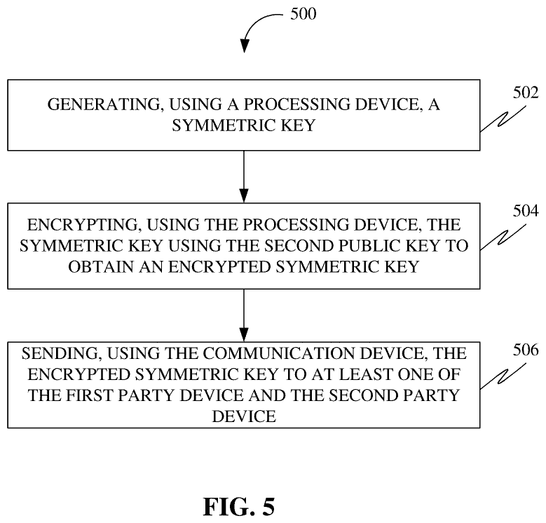

FIG. 5 is a flowchart of a method 500 to facilitate providing an encrypted symmetric key, in accordance with further embodiments. Accordingly, at 502, the method 500 may further include generating, using a processing device (such as the processing device 302), a symmetric key.

Further, at 504, the method 500 may include encrypting, using the processing device, the symmetric key using the second public key to obtain an encrypted symmetric key.

Further, at 506, the method 500 may include sending, using the communication device, the encrypted symmetric key to one or more of the first party device and the second party device.

In some embodiments, the method 500 may further include sending, using the communication device, the medical imaging data to the first party device. Further, the first party device may decrypt the medical imaging data with the first private key, may re-encrypt the medical imaging data with the symmetric key to obtain a re-encrypted imaging data, may digitally sign the re-encrypted imaging data and/or may send the re-encrypted imaging data to the second party device. Further, in some embodiments, the second party device may decrypt the encrypted symmetric key using a second private key associated with the second party device to obtain a decrypted symmetric key. Further, in some embodiments, the second party device may decrypt the medical imaging data using the decrypted symmetric key.

Further, in some embodiments, the method 500 may include recording, using the processing device, one or more transactions in blockchain. Further, the one or more transactions may include receiving the request from the second party device, transmitting the medical imaging data to the second party device, sending the re-encrypted imaging data to the second party device and sending the encrypted symmetric key to one or more of the first party device and the second party device.

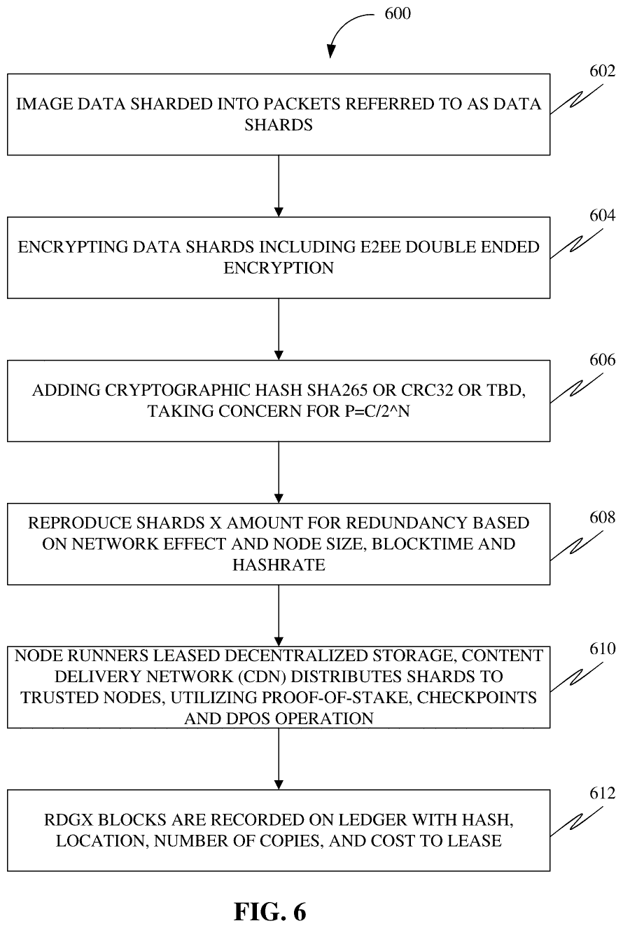

FIG. 6 is a flowchart of a method 600 to facilitate functioning of RDGX blockchain, in accordance with some embodiments. Accordingly, at 602, the method 600 may include a step of fragmenting image data (e.g. the medical imaging data) into packets that may be referred to as data shards. Further, at 604, the method 600 may include encryption of data shards. Further, the encryption of data shards may include end-to-end encryption (E2EE double ended encryption). Further, at 606, the method 600 may include adding cryptographic hash by using secure hash algorithms such as SHA256, cyclic redundancy check CRC32, or TBD. Further, the cryptographic hash may be added by taking concern for P=C/2\hat{ }N. Further, at 608, the method 600 may include a step of reproducing shards X amount for redundancy based on network effect and node size, blocktime and hashrate. For instance, the X amount may be equivalent to a ratio of network to the blocktime. Further, at 610, “node runners” incentivized (leased) decentralized storage. Further, a content delivery network (CDN) may distribute shards to trusted nodes, utilizing proof-of-stake, checkpoints and delegated proof-of-stake (DPOS) operation. Further, at 612, the method 600 may include recording RDGX blocks on ledger with hash, location, number of copies, and cost to lease.

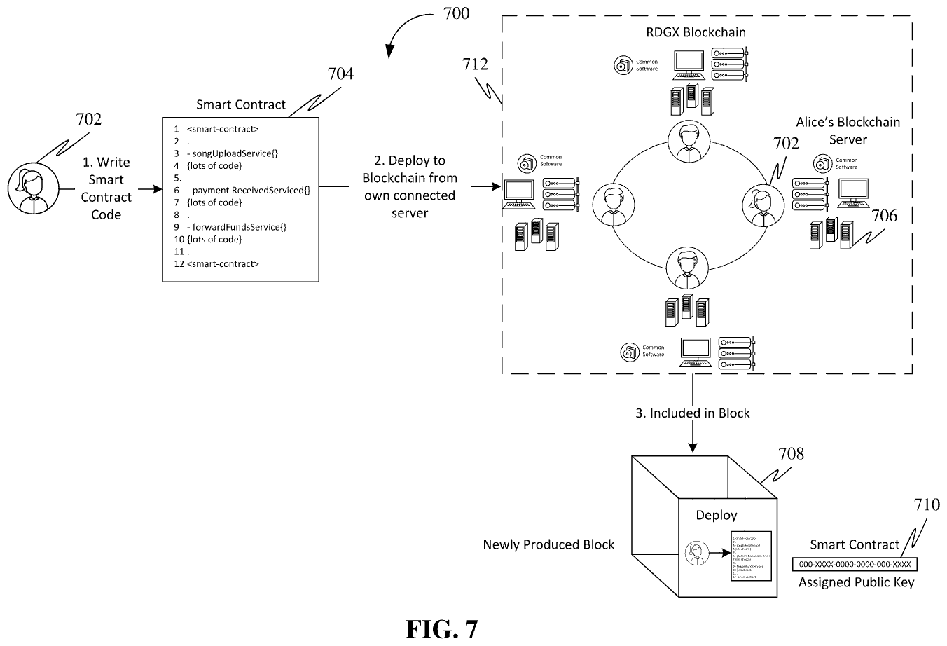

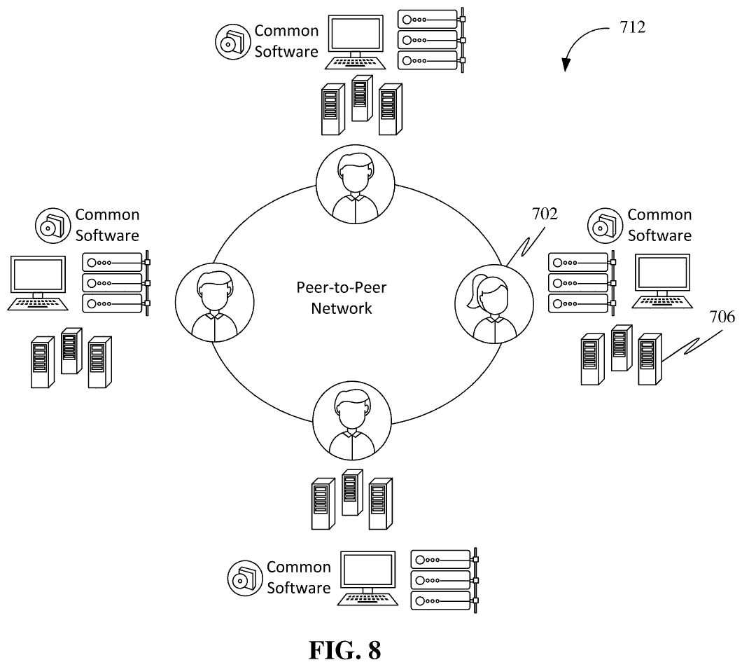

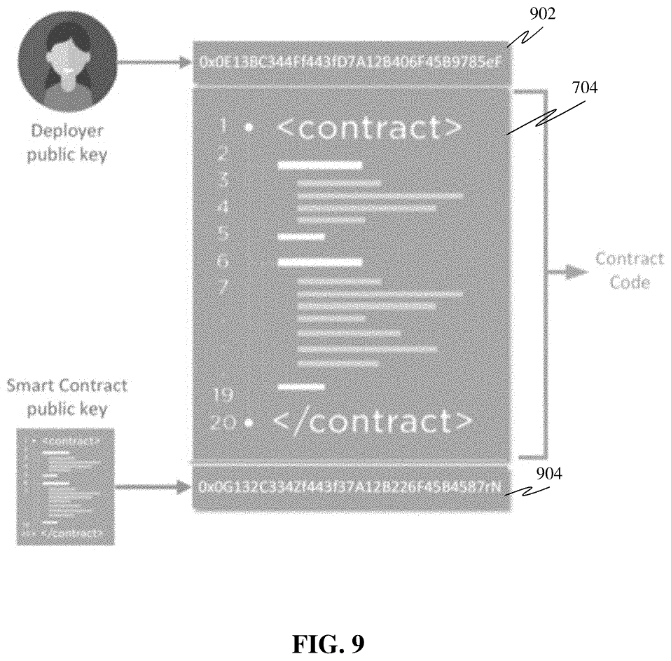

FIG. 7 is an exemplary representation of a system 700 facilitating functioning of RDGX blockchain in accordance with some embodiments. Accordingly, a user (such as Alice 702), in an instance, may write smart contract code (such as a smart contract 704). Further, the smart contract 704, in an instance, may be a computer protocol intended to digitally facilitate, verify, and/or enforce contracts in order to perform credible transactions without third parties. Further, the smart contract 704, in an instance, may be deployed to an RDGX blockchain 712. Further, the RDGX blockchain 712, in an instance, may be a peer to peer network of one or more servers (e.g. including Alice's server 706) corresponding to one or more users (such as Alice 702). FIG. 8 shows the RDGX blockchain 712 in further details. Further, once the smart contract 704 may be deployed, a block (such as a newly produced block 708) may be produced and a public key 710, in an instance, may be assigned for the smart contract 704. Further, FIG. 9 is an exemplary representation of a smart contract code (such as the smart contract 704) with a deployer public key 902 and a smart contract public key 904, in accordance with some embodiments.

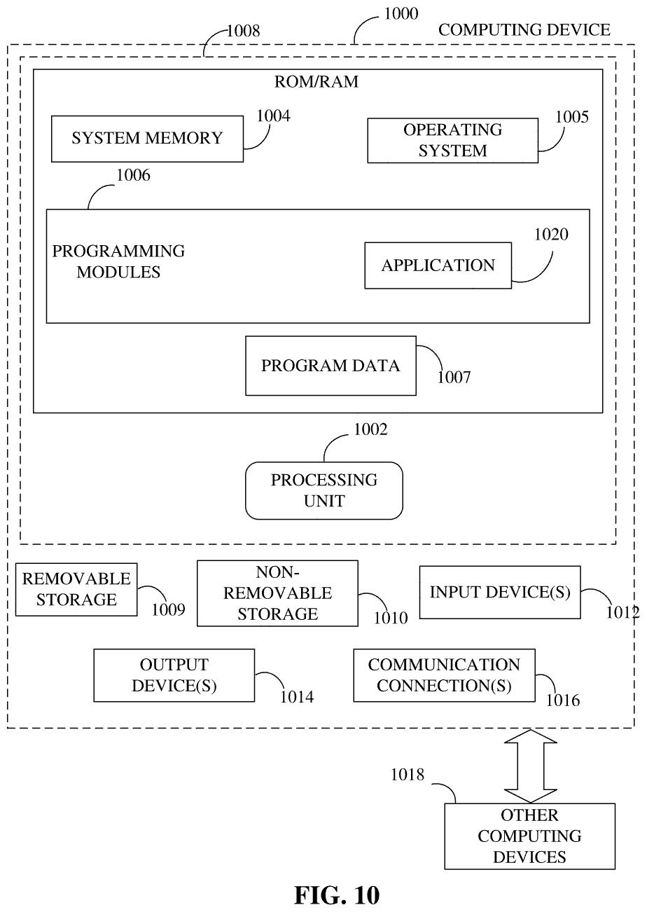

With reference to FIG. 10, a system consistent with an embodiment of the disclosure may include a computing device or cloud service, such as computing device 1000. In a basic configuration, computing device 1000 may include at least one processing unit 1002 and a system memory 1004. Depending on the configuration and type of computing device, system memory 1004 may comprise, but is not limited to, volatile (e.g. random-access memory (RAM)), non-volatile (e.g. read-only memory (ROM)), flash memory, or any combination. System memory 1004 may include operating system 1005, one or more programming modules 1006, and may include a program data 1007. Operating system 1005, for example, may be suitable for controlling computing device 1000′s operation. In one embodiment, programming modules 1006 may include image-processing module, machine learning module. Furthermore, embodiments of the disclosure may be practiced in conjunction with a graphics library, other operating systems, or any other application program and is not limited to any particular application or system. This basic configuration is illustrated in FIG. 10 by those components within a dashed line 1008.

Computing device 1000 may have additional features or functionality. For example, computing device 1000 may also include additional data storage devices (removable and/or non-removable) such as, for example, magnetic disks, optical disks, or tape. Such additional storage is illustrated in FIG. 10 by a removable storage 1009 and a non-removable storage 1010. Computer storage media may include volatile and non-volatile, removable and non-removable media implemented in any method or technology for storage of information, such as computer-readable instructions, data structures, program modules, or other data. System memory 1004, removable storage 1009, and non-removable storage 1010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1000. Any such computer storage media may be part of device 1000. Computing device 1000 may also have input device(s) 1012 such as a keyboard, a mouse, a pen, a sound input device, a touch input device, a location sensor, a camera, a biometric sensor, etc. Output device(s) 1014 such as a display, speakers, a printer, etc. may also be included. The aforementioned devices are examples and others may be used.

Computing device 1000 may also contain a communication connection 1016 that may allow device 1000 to communicate with other computing devices 1018, such as over a network in a distributed computing environment, for example, an intranet or the Internet. Communication connection 1016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1004, including operating system 1005. While executing on processing unit 1002, programming modules 1006 (e.g., application 1020 such as a media player) may perform processes including, for example, one or more stages of methods, algorithms, systems, applications, servers, databases as described above. The aforementioned process is an example, and processing unit 1002 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

1. A method of facilitating data management of medical imaging data, the method comprising:
receiving, using a communication device, a medical imaging data for a patient from a first party device associated with an Originating Radiologic Organization;
encrypting, using the first party device, the medical imaging data received from the first party device by using a first private key associated with the first party device:
storing, using a storage device, the medical imaging data on a blockchain;
receiving, using the communication device, a request from a second party device associated with a New Radiologic Organization to retrieve the medical imaging data, wherein the request received from the second party device includes a second public key associated with the second party device, wherein the request is to lease the medical imaging data for a period of time; and
securely transmitting, using the communication device, the medical imaging data to the second party device.

2. (canceled)

3. (canceled)

4. (canceled)

5. (canceled)

6. The method of claim 1 further comprising:
generating, using a processing device, a symmetric key;
obtaining an encrypted symmetric key by encrypting, using the processing device, the symmetric key using the second public key; and
sending, using the communication device, the encrypted symmetric key to at least one of the first party device and the second party device.

7. The method of claim 6 further comprising:
sending, using the communication device, the medical imaging data to the first party device; and
decrypting the medical imaging data with the first private key, obtaining a re-encrypted imaging data by re-encrypting the medical imaging data with the symmetric key, digitally signing the re-encrypted imaging data and sending the re-encrypted imaging data to the second party device by the first party device.

8. The method of claim 7 further comprising:
obtaining a decrypted symmetric key by decrypting the encrypted symmetric key using a second private key associated with the second party device by the second party device.

9. The method of claim 8 further comprising:
decrypting the medical imaging data using the decrypted symmetric key by the second party device.

10. The method of claim 9 further comprising:
recording, using the processing device, steps of receiving, using the communication device, the request from the second party device associated with the New Radiologic Organization to retrieve the medical imaging data, securely transmitting, using the communication device, the medical imaging data to the second party device, sending, using the communication device, the re-encrypted imaging data to the second party device and sending, using the communication device, the encrypted symmetric key to at least one of the first party device and the second party device.

11. A system of facilitating data management of medical imaging data, the system comprising:
a first party device associated with an Originating Radiologic Organization;
a second party device associated with a New Radiologic Organization;
a communication device;
a storage device;
wherein the communication device receives a medical imaging data for a patient from the first party device;
wherein the first party device encrypts the medical imaging data received from the first party device by using a first private key associated with the first party device:
wherein the storage device stores the medical imaging data on a blockchain;
wherein the communication device receives a request from the second party device to retrieve the medical imaging data;
wherein the request received from the second party device includes a second public key associated with the second party device;
wherein the request is to lease the medical imaging data for a period of time; and
wherein the communication device securely transmits the medical imaging data to the second party device.

12. (canceled)

13. (canceled)

14. (canceled)

15. (canceled)

16. The system of claim 11 further comprising:
a processing device;
wherein the processing device generates a symmetric key and obtaining an encrypted symmetric key by encrypting the symmetric key using the second public key; and
wherein the communication device receives the encrypted symmetric key from the processing device and sends the encrypted symmetric key to at least one of the first party device and the second party device.

17. The system of claim 16,
wherein the communication device sends the medical imaging data to the first party device, and
wherein the first party device decrypts the medical imaging data with the first private key, obtains a re-encrypted imaging data by re-encrypting the medical imaging data with the symmetric key, digitally signs the re-encrypted imaging data and sends the re-encrypted imaging data to the second party device.

18. The system of claim 17,
wherein the second party device obtains a decrypted symmetric key by decrypting the encrypted symmetric key using a second private key associated with the second party device.

19. The system of claim 18,
wherein the second party device decrypts the medical imaging data using the decrypted symmetric key.

20. The system of claim 19,
wherein the processing device records that the communication device receives the request from the second party device, securely transmits the medical imaging data to the second party device, sends the re-encrypted imaging data to the second party device and sends the encrypted symmetric key to at least one of the first party device and the second party device.
